(19) United States
(12) Patent Application Publication
(10) Pub. No.: US 2017/0035506 A1
(43) Pub. Date: Feb. 9, 2017

(54) Laser Diode Package Arrangement with Interchangable Tip

(71) Applicant: AMD Lasers, Indianapolis, IN (US)

(72) Inventor: Bart Waclawik, Indianapolis, IN (US)

(21) Appl. No.: 14/817,620

(22) Filed: Aug. 4, 2015

(51) Int. Cl. A61B 18/20

(52) U.S. Cl. CPC A61B 18/20; A61B 2018/0091

(57) ABSTRACT

A laser diode packaging arrangement for a laser system handpiece configured to generate laser power within the hand-piece using a TO-CAN diode. Instead of using optical connections, an electrical connection is made to the handpiece from a laser system body, where the laser diode generates laser power within a handpiece body to a detachable laser tip assembly. The detachable laser tip assembly allows for interchangeable laser tips to be employed to provide laser tips having the same or different geometries. By employing various electrical contacts within the hand-piece and the end applicator, the laser can auto-detect the type of tip attached to the system and configure itself.

### FIELD OF THE DISCLOSURE

The present disclosure relates to medical laser packaging. More specifically, the present disclosure relates to packaging laser diode arrangements to provide laser generation abilities within a laser handpiece.

### BACKGROUND

Light amplification by stimulated emission of radiation, or “LASER”, is a device that creates and amplifies electromagnetic radiation of specific wavelength through process of stimulated emission. In laser devices, all the light rays have three key properties: single wavelength, coherence (they travel in same direction), and same phase. A laser typically includes a gain medium, a mechanism to energize it, and something to provide optical feedback. The gain medium is typically a material with properties that allow it to amplify light by way of stimulated emission. Light of a specific wavelength that passes through the gain medium is amplified (increases in power). There are many laser classifications depending on the principle of operation and the medium. For instance, there are lasers where the gain medium is a gas (e.g. CO2), a crystal (e.g. Er:YAG), or a semiconductor (e.g. GaAlAr). This disclosure focuses specifically on semiconductor (diode) lasers.

Lasers have found numerous uses over the years, including industrial applications, measurement and medical applications. Lasers are also found in various dental, medical treatment, and surgical applications. Currently, many medical lasers are configured to have a light producing source, such as a laser diode, in the housing or body portion of the laser, where optical coupling is used to attach the laser source to a laser handpiece which delivers the energy to the patient. One of the drawbacks of these configurations is that optical fiber coupling is required to transmit optical energy from the body portion to the handpiece. Another drawback is that expensive and/or complicated optical handpiece attachments are needed for laser devices having disposable or interchangeable tips. What is needed is technology for packaging laser diodes for effective and efficient use with interchangeable and/or disposable handpiece tips.

### SUMMARY

Accordingly, under some illustrative embodiments, a laser system is disclosed, comprising a laser system body comprising power circuitry and a processor; and a handpiece, electrically coupled to the laser system body via an electrical cable, wherein the handpiece comprises a handpiece body and further comprises a laser diode configured to receive power from the electrical cable to produce laser energy within the handpiece body, a lens configured to focus the laser energy from the laser diode within the handpiece body, and a detachable laser tip assembly comprising a laser tip and laser tip body, wherein the laser tip body is configured to be coupled to the handpiece body and the laser assembly is configured to receive the laser energy focused from the lens to emit the received laser energy from the handpiece.

In other illustrative embodiments, a method is disclosed for operating a laser system, comprising electrically coupling a handpiece to a laser system body via an electrical cable, wherein the handpiece comprises a handpiece body coupled to a detachable laser tip assembly, and wherein the laser system body comprises power circuitry and a processor; providing power to a laser diode to produce laser energy within the handpiece body; focusing the produced laser energy within the handpiece body via a lens; and receiving and emitting the focused laser energy from the handpiece via a laser tip comprising a laser tip body configured in the detachable laser tip assembly.

In further illustrative embodiment, a laser handpiece, configured to be electrically coupled to a laser system body, is disclosed, comprising a handpiece body; a laser diode comprising electrical contacts configured to receive power from the laser system body electrical cable to produce laser energy within the handpiece body; a lens configured to focus the laser energy from the laser diode within the handpiece body, and a detachable laser tip assembly comprising a laser tip and laser tip body, wherein the laser tip body is configured to be detachably coupled to the handpiece body and the laser assembly is configured to receive the laser energy focused from the lens to emit the received laser energy from the handpiece.

### BRIEF DESCRIPTION OF THE FIGURES

The present disclosure will become more fully understood from the detailed description given herein below and the accompanying drawings which are given by way of illustration only, and which thus do not limit the present disclosure, and wherein:

FIG. 1 shows a medical laser device system that includes a body and a handpiece under an illustrative embodiment;

FIG. 2 shows a laser device handpiece comprising a laser diode integrated into the handpiece along with an optic lens, suitable for use in the laser device system of FIG. 1 under an illustrative embodiment; and

FIG. 3 shows a laser device handpiece comprising a laser diode integrated into the handpiece along with an optic lens and strain relief suitable for use in the laser device system of FIG. 1 under another illustrative embodiment;

FIG. 4 shows a diode suitable for use in any of the embodiments of FIGS. 2-3 arranged as a TO-CAN laser diode under an illustrative embodiment;

FIG. 5 shows a laser device handpiece having a curing tip comprising a laser diode integrated into the handpiece along with an optic lens and strain relief suitable for use in the laser device system of FIG. 1 for curing applications under another illustrative embodiment; and

FIG. 6 shows a battery-powered portable laser device handpiece having a curing tip comprising a laser diode integrated into the handpiece along with an optic lens and strain for curing applications under another illustrative embodiment.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

Exemplary embodiments are provided throughout so that this disclosure is sufficiently thorough and fully conveys the scope of the disclosed embodiments to those who are skilled in the art. Numerous specific details are set forth, such as examples of specific components, devices, and methods, to provide this thorough understanding of embodiments of the present disclosure. Nevertheless, it will be apparent to those skilled in the art that specific disclosed details need not be employed, and that exemplary embodiments may be embodied in different forms. As such, the exemplary embodiments should not be construed to limit the scope of the disclosure. In some exemplary embodiments, well-known processes, well-known device structures, and well-known technologies may not be described in detail.

The terminology used herein is for the purpose of describing particular exemplary embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The steps, processes, and operations described herein are not to be construed as necessarily requiring their respective performance in the particular order discussed or illustrated, unless specifically identified as a preferred order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the exemplary embodiments.

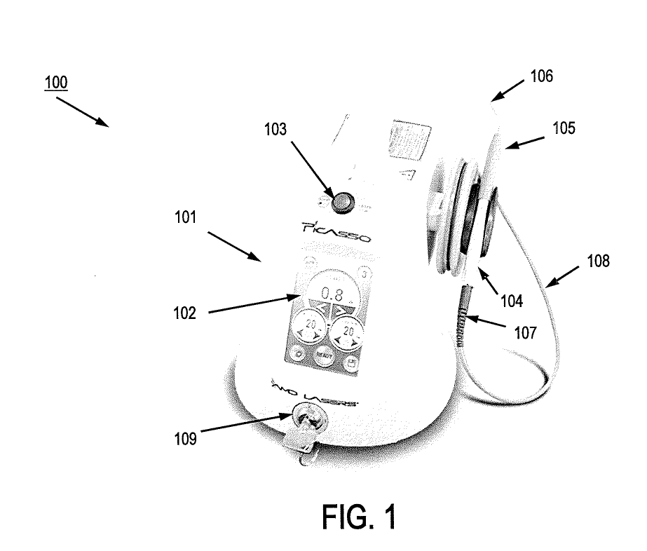

Turning now to FIG. 1, a laser system 100 is shown under an illustrative embodiment, comprising a body 101, touchscreen 102 and power button 103. In some illustrative embodiments, system body 101 may be detachable and locked into position via lock 109 as shown in the figure. As is known in the art, laser system body 101 comprises electronic circuitry, such as one or more processors, microcontrollers, memory, power circuitry and other electronic circuitry for providing power and controlling operation of medical laser system 100. Accordingly, such components are not explicitly shown in FIG. 1 for the sake of brevity.

Laser system body 101 may be electrically coupled to handpiece 104 via electrical cable 108, and may further comprise a handpiece strain relief portion 107. Handpiece 104 may also include a removable top housing portion 105 that is coupled to an optical laser tip that may be disposable. While in preferred embodiments the system body 101 is electrically coupled to handpiece 104 via electrical cable 108, it is possible to include optical wire and connections within the electrical cable 108 to accommodate alternate configurations.

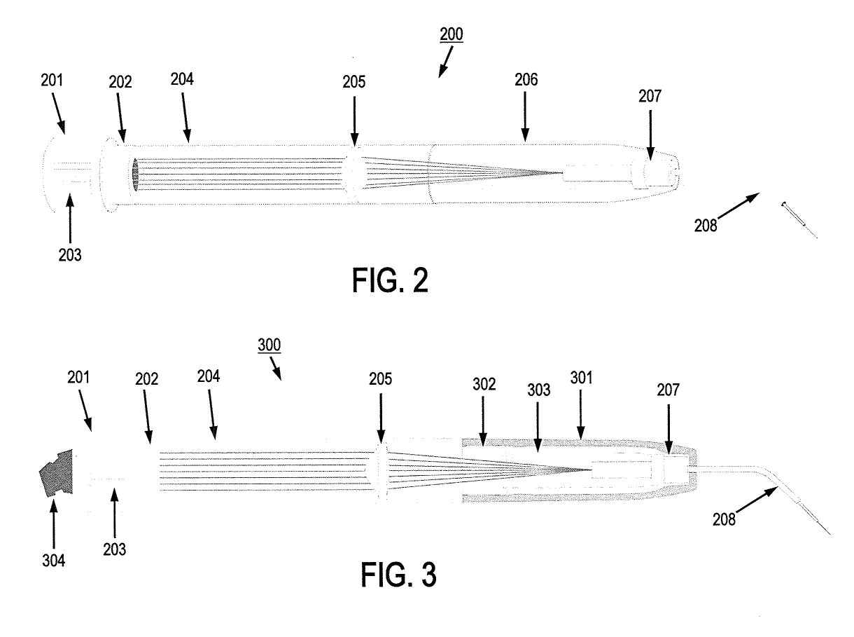

Turning to FIG. 2, a handpiece 200 is disclosed that is suitable for use as a handpiece (104-106) of laser system 100 illustrated in FIG. 1 under an illustrative embodiment. In this example, handpiece 200 includes a handpiece base 201 that surrounds a laser diode 202 that may include electrical contacts 203 configured as pins extending within handpiece base 201. It should be understood by those skilled in the art that other types of electrical contacts, such as sockets, plate contacts, spring contacts, and the like are contemplated in the present disclosure. The electrical contacts 203 may be configured to couple with a complimentary plug (not shown) that is attached to an electrical cable (e.g., electrical cable 108), where power and/or control signals from the laser system body may be provided to laser diode 202.

Since electrical connections are used, an advantageous configuration may be achieved for allowing interchangeable handpieces. In some illustrative embodiments, the processor(s) within a laser system body 101 may automatically detect a type of laser tip (e.g., curing, surgical, periodontal, therapeutic) that is attached to the laser system body 101 according to the number of rings 608 making contact. In other illustrative embodiments, laser diode 202 may be configured with additional circuitry, including, but not limited to internal switches or jumpers for automatic identification of a handpiece type. In one example such identification may occur using a low-power signal from the laser system body 101 to the laser diode 202, where the identification is made upon processing a characteristic of the signal (e.g., voltage, current, resistance, etc.) returning from the laser diode 202.

The front (or illuminating) face of laser diode 202 is configured to couple to a handpiece body 204, which may be manufactured from a metal with high thermal conductivity (e.g., aluminum, copper), or other suitable material and act as a radiator from the laser diode 202. During operation, laser energy is transmitted from laser diode 202 to lens 205. In one illustrative embodiment, lens 205 is configured as an aspheric lens positioned in front of laser diode 202. In this example, the aspheric lenses allows for correction of spherical aberration, which provides better quality collimated beams and a smaller spot size, particularly for medical applications. A further collimator assembly may be provided or integrated with aspheric lens 205 to provide even more focused beams. The lens 205 may be threaded or otherwise configured to position the lens 205 at a predetermined proximal distance between the laser diode 202 and optical base 207 of laser-emitting tip 208.

In one example, laser diode 202 is affixed to handpiece body 204. In another example, laser diode 202 may be detachable from handpiece body 204 to allow handpiece bodies of different lengths and/or containing different kinds of lenses to be interchangeably attached to laser diode 202. Such a configuration would be advantageous in allowing a user to efficiently customize and adjust focal lengths and/or aspheric effect of a given lens, and thus providing greater flexibility in producing a desired type of laser energy for a given application.

The handpiece body 204 may be coupled to a tip housing 206 that holds an optical tip that may include an optical tip base 207 and laser-emitting tip 208. During use, the lens 205 may be configured to focus laser energy into the optical tip base 207 and emitted externally via laser-emitting tip 208. In one example, the tip housing 206 may be affixed to the handpiece body 204. In another example, the tip housing 206 may be detachable via threads, positive lock, or any other suitable mechanism. In a still further example, only the laser-emitting tip 208 is configured to be detachable. The detachable tip configuration may be advantageous for providing optical tips of different geometries for providing laser energy in a specific focus, shape, pattern etc. Of course, when multiple laser applications of the same type are used, the removable tip 208 or tip housing 206 may be disposable to provide a clean environment for patients after each use. Since electrical connection is used between the handpiece 200 and the laser body 100, the laser is able to detect the type of tip based on the electrical connections.

Turning to FIG. 3, another illustrative embodiment is shown for a handpiece 300 that is similar to the handpiece 200 discussed above in connection with FIG. 2. In this example, handpiece 300 includes a handpiece base 201 that surrounds a laser diode 202 that may include electrical contacts 203 configured as pins extending within handpiece base 201. As with the embodiment of FIG. 2, it should be understood by those skilled in the art that other types of electrical contacts, such as sockets, plate contacts, spring contacts, and the like are contemplated in the present disclosure. The electrical contacts 203 may be configured to couple with a complimentary plug (not shown) that is attached to an electrical cable (e.g., electrical cable 104), where power and/or control signals from the laser system body may be provided to laser diode 202. In the embodiment of FIG. 3, a strain relief portion 304 may be provided to relieve excessive wear on electrical cable 104.

Again, since electrical connections are used, an advantageous configuration may be achieved for allowing interchangeable handpieces, where the processor(s) within a laser system body 101 may automatically detect a type of tip diameter of tip type (e.g. surgical tip, periodontal tip, curing tip, whitening tip, etc.) that is attached to the laser system body 101 similar to the embodiment in FIG. 2. The front face of laser diode 202 is configured to couple to a handpiece body 204, which may be manufactured from a metal or other suitable material and act as a radiator from the laser diode 202 and may also include an insulating cover or material. In an illustrative embodiment, the handpiece body 204 may extend (302, 303) to accommodate a laser tip base 207 at a distal end that hold laser tip 208, and may be encased in the laser tip cover 301, which may be permanently or detachably affixed to the handpiece bode via attachment portion 302.

During operation, laser energy is transmitted from laser diode 202 to lens 205 which may be configured as an aspheric lens positioned in front of laser diode 202. The lens 205 may be threaded or otherwise configured to position the lens 205 at a predetermined proximal distance between the laser diode 202 and optical base 207 of laser-emitting tip 208. In one example, laser diode 202 is affixed to handpiece body 204. In another example, laser diode 202 may be detachable from handpiece body 204 to allow handpiece bodies of different lengths and/or containing different kinds of lenses to be interchangeably attached to laser diode 202.

In the example of FIG. 3, the handpiece body 204 may be coupled to a tip housing 301 that encloses an optical tip that may include an optical tip base 207 and laser-emitting tip 208. During use, the lens 205 may be configured to focus laser energy into the optical tip base 207 and emitted externally via laser-emitting tip 208. As mentioned previously, the tip housing 301 may be coupled to the handpiece body 204 via attachment portion 302. The coupling of the tip housing 301 to the handpiece body 304 may be permanent or detachable using threads, positive lock, or any other suitable mechanism for attachment portion 302.

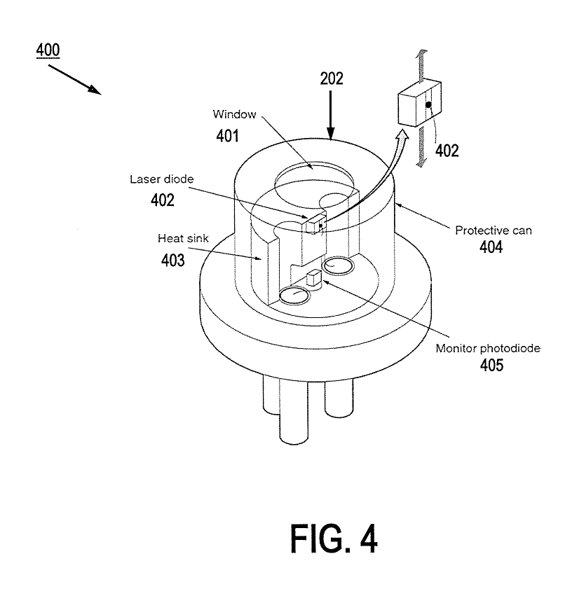

In one illustrative embodiment, laser diode 202 may be configured in a transistor outline (“TO-CAN”) package 400 shown in FIG. 4. In this example, laser diode 202 (also shown in exploded view in FIG. 2) is coupled to a heat sink 403 and monitor photodiode 405 that may produce current proportional to the output laser diode 202 optical power. As shown in the figure, the laser diode 202, heat sink 403 and monitor photodiode 405 are encased in a protective can 404, with a window 401 configured in the top of protective can 404 to allow laser light to be emitted out. The TO-CAN package may be configured as 5.6 mm or 9 mm diameter, having power suitable for a variety of medical procedures (typically on an order of 500 mW-10 W). However, it should be appreciated by those skilled in the art that other suitable diameters and powers are contemplated in the present disclosure.

It should be appreciated by those skilled in the art that electrically coupling a laser handpiece to a laser system body and providing laser generation capabilities (e.g., via laser diode 202) in the handpiece provides greater flexibility to a user in configuring laser systems to a particular application. Furthermore, by providing interchangeable or disposable tips in the handpiece, users may more quickly and efficiently configure or re-configure a laser system physically using only the handpiece. By providing exchangeable fiber tips, special and optimized geometries may be realized for various applications using different intensity distributions and intensity profiles.

For example, spherical or hemispherical tip ends may be used to diffuse laser energy provided by diode 202. In another example, conically-shaped tips (or “tapers”) may be used for cutting applications. In a still further example, tips of different core diameter may be used to perform a variety of surgical and non-surgical procedures. Effects of laser may be dependent on the type of laser used, the intensity of the laser, and the type of tissue the laser is being applied to, since a particular tissue may transmit, absorb, scatter or reflect the laser light.

Under the present disclosure, the interchangeable tip configurations may be used to provide use of various types of laser emissions in medical diagnosis, treatment, or therapy. Using the processor and power circuitry contained within laser system body 101 (and controlled via touch screen 102), higher or lower power may be applied to a handpiece (e.g., 200, 300) depending on the diode (202), handpiece body (204) configuration, and laser tip (208) geometry. When a laser diode 202 is configured as a high-power laser, the system 100 may be configured to use laser energy to produce heat. As a result, such an application generates tissue interaction effects through thermal processes. These effects can include vaporization of tissue, coagulation, cauterization, and carbonization. In some illustrative embodiments, the high-power laser may have an output power of more than 500 mW. For lower-power (intermediate) application, therapeutic effects may be provided without producing significant heat. In some illustrative embodiments, the lower-power laser may have an output power ranging from 100-500 mW. For low-power applications the lasers typically will have photo-biostimulive effects or photo-biochemical reactions. The output power of these lasers may be less than 250 mW. In addition to power, interchangable laser tips may be applied for various laser applications including, but not limited to, incisions, vaporization, or coagulation, where different laser tips may provide various energy fluence effects.

By providing easily interchangeable diode types via the handpiece, a single laser housing (e.g., 101) may be used to provide a multitude of different laser wavelengths. Lasers within the ultraviolet region (100 to 38 nm) are able to ionize tissues, a process known as photochemical desorption. Lasers of longer wavelengths, especially those within the infrared part of the spectrum (700 to 10,000 nm), cause significant tissue heating. Additionally, lasers using red, yellow, green, blue, violet and other colors are advantageous for emitting visible electromagnetic radiation, typically in the 360 nm to 600 nm range to target or point a spot on a surface subjected to medical, surgical and/or therapeutic applications.

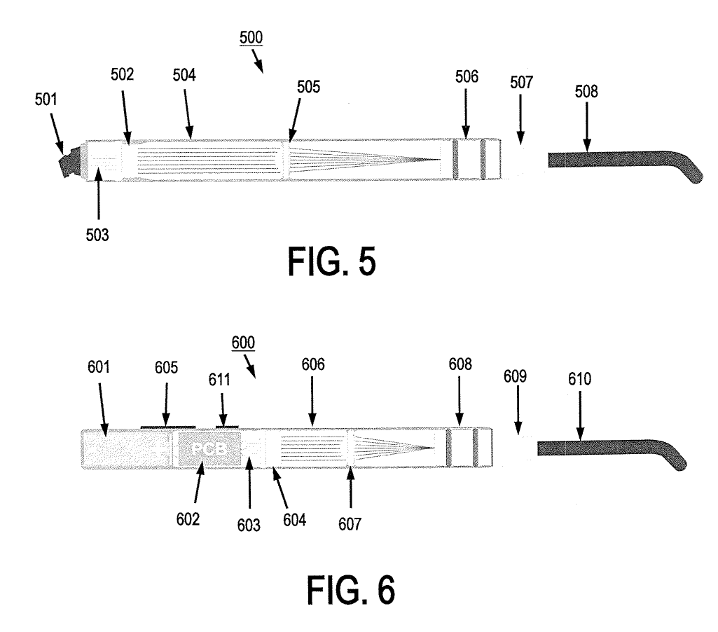

Turning to FIG. 5, another illustrative embodiment is shown for a laser handpiece 500 configured for curing applications including, but not limited to, composite curing. Laser handpiece 500 may be configured similarly to any of the handpieces disclosed herein and may include a handpiece body 205 coupled to a tip housing 507 that encloses an optical tip that may include an optical tip base 506 and laser-emitting tip 508 which, in one example, is a curing connector (506) and curing light guide (508). The curing light may be used for polymerization of light cure resin based composites and may be configured for any of several different dental materials that are curable by light. In some embodiments, the diode 502 may be configured receive power provided to terminals 503 to produce light under the visible blue light spectrum. This light may delivered over a range of wavelengths. In one embodiment, a strain relief 503 may be provided to secure and protect a coupled power cord (e.g., 108).

During use, the lens 505 may be configured to focus laser energy into the optical tip base 506 and emitted externally via laser-emitting tip 508. As mentioned previously, the tip housing 507 may be coupled to the handpiece body 505 via base connector portion 506. The coupling of the tip housing 507 to the handpiece body 504 may be permanent or detachable using threads, positive lock, or any other suitable mechanism for attachment portion 506. The attachment 506 will feature mechanical and electrical features that will allow the laser system 100 to auto-determine that a curing tip was inserted.

While embodiments discussed above were discussed in connection with a laser system body (e.g. laser system body 101), other embodiments may include a stand-alone laser handpiece 600, such as the illustrative embodiment shown in FIG. 6. In this example, handpiece body 606 houses a portable power supply 601, such as a battery or cell that is operatively coupled to printed circuit board 602, which may comprise processor(s), power circuitry, for providing and controlling power provided to laser diode 604 via laser diode. A miniature display 605, which may be controlled by circuit board 602, and button 611 may be provided to allow activation, control and visual monitoring of laser handpiece 600 operations. In some illustrative embodiments, display 605 may be equipped with miniaturized speakers for audio output.

One or more outputs from circuit board 602 may be provided to laser diode 605 via laser diode terminals 603, wherein light from laser diode 605 is provided to lens 607 to focus laser energy into the optical tip base 608 and emitted externally via laser-emitting tip 610. The tip housing 609 may be coupled to the handpiece body 606 via base connector portion 608. The coupling of the tip housing 609 to the handpiece body 606 may be permanent or detachable using threads, positive lock, or any other suitable mechanism for attachment portion 608.

In the foregoing detailed description, it can be seen that various features are grouped together in individual embodiments for the purpose of brevity in the disclosure. This method of disclosure is not to be interpreted as reflecting an intention that the subsequently claimed embodiments require more features than are expressly recited in each claim.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather are to be accorded the widest scope consistent with the principles and novel features disclosed herein.

What is claimed is:

1. A laser system, comprising:
a laser system body comprising power circuitry and a processor; and
a handpiece, electrically coupled to the laser system body via an electrical cable, wherein the handpiece comprises a handpiece body and further comprises:
a laser diode configured to receive power from the electrical cable to produce laser energy within the handpiece body,
a lens configured to focus the laser energy from the laser diode within the handpiece body, and
a detachable laser tip assembly comprising a laser tip and laser tip body, wherein the laser tip body is configured to be coupled to the handpiece body and the laser assembly is configured to receive the laser energy focused from the lens to emit the received laser energy from the handpiece.

2. The laser system of claim 1, wherein the laser diode comprises a transistor outline diode package.

3. The laser system of claim 1, wherein the lens comprises an aspheric lens.

4. The laser system of claim 1, wherein the detachable laser tip assembly is configured to release the laser tip and receive another laser tip.

5. The laser system of claim 4, wherein the another laser tip comprises a different geometry from the laser tip.

6. The laser system of claim 1, wherein the processor is configured to determine at least one characteristic of the electrically coupled handpiece and determine the type of the tip attached.

7. The laser system of claim 1, wherein the laser diode is detachably coupled to the handpiece body.

8. A method for operating a laser system, comprising:
electrically coupling a handpiece to a laser system body via an electrical cable, wherein the handpiece comprises a handpiece body coupled to a detachable laser tip assembly, and wherein the laser system body comprises power circuitry and a processor;
providing power to a laser diode to produce laser energy within the handpiece body;
focusing the produced laser energy within the handpiece body via a lens; and
receiving and emitting the focused laser energy from the handpiece via a laser tip comprising a laser tip body configured in the detachable laser tip assembly.

9. The method of claim 8, wherein the laser diode comprises a transistor outline diode package.

10. The method of claim 8, wherein the lens comprises an aspheric lens.

11. The method of claim 8, wherein the detachable laser tip assembly is configured to release the laser tip and receive another laser tip.

12. The method of claim 11, wherein the another laser tip comprises a different geometry from the laser tip.

13. The method of claim 8, further comprising determining, via the processor, at least one characteristic of the electrically coupled handpiece.

14. The method of claim 8, wherein the laser diode is detachably coupled to the handpiece body.

15. A laser handpiece, configured to be electrically coupled to a power supply, comprising:
a handpiece body;
a laser diode comprising electrical contacts configured to receive power from the power supply to produce laser energy within the handpiece body;
a lens configured to focus the laser energy from the laser diode within the handpiece body; and
a detachable laser tip assembly comprising a laser tip and laser tip body, wherein the laser tip body is configured to be detachably coupled to the handpiece body and the laser assembly is configured to receive the laser energy focused from the lens to emit the received laser energy from the handpiece.

16. The laser system of claim 15, wherein the laser diode comprises a transistor outline diode package.

17. The laser system of claim 15, wherein the lens comprises an aspheric lens.

18. The laser system of claim 15, wherein the detachable laser tip assembly is configured to release the laser tip and receive another laser tip having a different geometry from the laser tip.

19. The laser system of claim 15, wherein the processor is configured to determine at least one characteristic of the electrically coupled handpiece and the type of the tip attached to the handpiece and auto-configure the laser based on the type of tip inserted.

20. The laser system of claim 15, wherein the power supply is housed within the handpiece body.
